(19) United States
(12) Patent Application Publication
(10) Pub. No.: US 2023/0211086 A1
(43) Pub. Date: Jul. 6, 2023

(54) DEVICE FOR HOMOGENEOUS INJECTION OF MEDICINE

(71) Applicant: JUVENUI Co., LTD, Seongnam-si, Gyeonggi-do (KR)

(72) Inventor: Hyun Hong KIM, Seoul (KR)

(73) Assignee: JUVENUI Co., LTD, Seongnam-si, Gyeonggi-do (KR)

(21) Appl. No.: 18/076,362

(22) Filed: Dec. 6, 2022

(30) Foreign Application Priority Data
Dec. 30, 2021 (KR) 10-2021-0192394

(51) Int. Cl. A61M 5/24; A61M 5/315

(52) U.S. Cl. CPC A61M 5/24; A61M 5/31515; A61M 5/31546; A61M 2205/3331

(57) ABSTRACT

Provided herein is a system for homogeneous injection of medicine including a device for homogeneous injection of medicine, which may include a cylinder holding part having a cylinder of a syringe held therein, a plunger resting part being configured to be independent from the cylinder holding part and having an end of the plunger of the syringe rest therein, first and second pressurized lead screw supporting parts each being provided on one side of the cylinder holding part and one side of the plunger resting part, respectively, and being connected to a pressurized lead screw being provided with a screw thread, and a motor being connected to one side of the first pressurized lead screw supporting part so as to rotate the pressurized lead screw; and a monitoring unit monitoring injection dosage and having one side connected to a needle of the syringe or a medicine outlet of the syringe cylinder and having its opposite side connected to a tube providing medicine to a patient, thereby monitoring physical quantities related to the injection of the medicine.

### CROSS-REFERENCE TO RELATED APPLICATIONS

This application claims priority to Korean Patent Application 10-2021-0192394, filed on Dec. 30, 2021, in the Korean Intellectual Property Office, the disclosure of which is incorporated herein by reference in its entirety.

### TECHNICAL FIELD

The present invention relates to a device for monitoring injection dosage of medicine and, most particularly, to a device for homogeneous injection of medicine that may increase medicinal effects resulting from homogeneous injection and reduce burden that a patient may need to endure due to non-homogeneous injection by meticulously controlling an injection system of medicine, wherein dosage of the medicine is required to be most strictly regulated.

### BACKGROUND ART

A syringe includes a needle being attached in order to inject medication into a patient's body, a cylinder supporting the needle and accommodating the medication, and a plunger performing reciprocating movements within the cylinder so as to discharge the medication outside of the cylinder or to introduce the medication from the outside and into the cylinder. There also exists another type of injection method that uses an intravenous (IV) bag set. The IV bag set includes a needle invading a patient's body, a tube being connected to the needle, a pouch accommodating medication so as to allow the medication to be injected through the tube, and a valve being connected to the tub and provided so as to allow injection dosage of the medication to be regulated.

In case of the plunger of a syringe, the injection dosage is regulated by controlling pressure applied to the plunger by a force of fingers pushing (or pressing) the plunger. Herein, however, when regulating the injection dosage by adjusting the pushing force of the fingers, the control of pressure may not be consistent, which may lead to a difficulty in homogenous (or consistent) quantization of injection dosage based on a unit time for injecting medication. That is, when it is assumed that a dosage of 600 ml per hour should be injected, although the unit time may be defined, in theory, as injecting a dosage of 10 ml per minute, since the size of a cross-sectional surface of the tube, through which the medication passes (or flows), is being adjusted by using the valve or a dial, it is almost impossible to actually quantify a dosage of 10 ml per minute accurately and homogenously.

Meanwhile, in the Korean Utility Model Registration No. 0486792 “Medication injection device for a syringe”, according to an aspect of the medication injection device for a syringe, the medication injection device for a syringe comprising a cylinder having medication supplied therein, and a piston being installed inside the cylinder so as to perform linear movements, thereby allowing the medication to be supplied inside the cylinder or allowing the medication supplied inside the cylinder to be discharged outside of the cylinder for injection by performing linear movements with respect to the cylinder, may include a housing that configures a resting space for the cylinder to rest and a sliding space being divided from the resting space, a sliding member being installed to be linearly moveable along the sliding space and being fixed to the piston, an elastic member for injection being extended, when the piston performing a linear movement with respect to the cylinder so that medication can be supplied inside the cylinder, in a state where the sliding member is fixed to the piston, and providing elasticity to the piston through the sliding member, so as to allow the medication supplied inside the cylinder to be discharged outside of the cylinder for injection, and a locking part optionally allowing movement of the sliding member with respect to the housing, in a state where the elastic member for injection is extended.

Although the aforementioned prior art is appropriate for resolving the problems lying in the related art technology, in that homogeneous (or consistent) pressure can be applied to the piston, equipment error may occur in the prior art. And, accordingly, there lies a problem in that is may be difficult to recognize (or determine) the occurrence or non-occurrence of such equipment error. That is, in a case where strictly homogeneous injection is required, if the homogeneous injection fails to be performed due to an equipment error, in some cases, there lies a problem in that such failure may have a critical impact on the patient (or the patient's health). Furthermore, there also exists a danger in making an error of assuming that the injection dosage will be homogeneously (or consistently) maintained as long as the pressure applied by the elastic member is consistently maintained.

### DETAILED DESCRIPTION OF THE INVENTION

### Technical Problem

The present invention has been devised to resolve the above-described problems, and, accordingly, an object of the present invention is to provide a means for monitoring homogeneous injection of medicine, so that a moving distance of the plunger of a syringe for homogeneous injection of medicine can be consistently maintained.

Additionally, another object of the present invention is to compare information on the injection dosage (or flow rate) of medicine, which is measured through a monitoring means that is provided in order to strictly maintain the homogeneousness (or homogeneity or consistency) in the injection dosage, with a configured total volume of an injection pump or an injection dosage (or flow rate) per unit time, and, when needed, correcting the injection dosage by adjusting the moving distance of the plunger.

That is, the other object of the present invention is to provide a monitoring means that can precisely regulate the moving distance and rate of the plunger based on the injection dosage, based on an assumption that a consistent moving distance and pressure of the plunger do not mandatorily determine a homogeneous quantization of the injection dosage (or flow rate).

Technical problems to be solved by the present invention are not limited to the above-mentioned technical problems, and a person having ordinary skill in the art may apparently understand other technical problems not mentioned herein, based on the descriptions according to the present invention.

### Technical Solution

In order to achieve the above-described technical objects of the present invention, provided herein is a system for homogeneous injection of medicine including a device for homogeneous injection of medicine, which may include a cylinder holding part having a cylinder of a syringe held therein, a plunger resting part being configured to be independent from the cylinder holding part and having an end of the plunger of the syringe rest therein, first and second pressurized lead screw supporting parts each being provided on one side of the cylinder holding part and one side of the plunger resting part, respectively, and being connected to a pressurized lead screw being provided with a screw thread, and a motor being connected to one side of the first pressurized lead screw supporting part so as to rotate the pressurized lead screw; and a monitoring unit monitoring injection dosage and having one side connected to a needle of the syringe or a medicine outlet of the syringe cylinder and having its opposite side connected to a tube providing medicine to a patient, thereby monitoring physical quantities related to the injection of the medicine.

Preferably, the system for homogeneous injection of medicine may further include a gear set being connected to one side of the motor or first pressurized lead screw supporting part, or being provided inside the first pressurized lead screw supporting part, so as to control the rotation of the pressurized lead screw.

Preferably, rotation of the pressurized lead screw may be accurately controlled by configuring the gear set of a primary gear and a secondary gear, wherein a diameter of the secondary gear may be greater than a diameter of the primary gear, and by configuring the pressurized lead screw to be connected to the secondary gear, so as to be capable of performing vertical or horizontal movements according to a number of rotations of the secondary gear.

Preferably, the monitoring unit may be equipped to the device for homogeneous injection of medicine.

Preferably, the monitoring unit may be connected to a control unit controlling rotation of the motor provided in the device for homogeneous injection of medicine according to a medication injection situation of the device.

Preferably, the monitoring unit may include a pressure measuring unit measuring pressure on the plunger caused by the device for homogeneous injection of medicine or outlet pressure of the medicine, a flow rate measuring unit measuring a flow rate (or injection dosage) of the medicine injected by the device for homogeneous injection of medicine, and an air measuring unit measuring whether or not air is included in the medicine.

Preferably, the monitoring unit may be detachably connected to the device for homogeneous injection of medicine.

Preferably, the cylinder holding part and the plunger resting part may be connected by a guide, and the plunger resting part may move along a horizontal direction to a vertical shaft of the guide, wherein the guide is used as a medium, according to the rotation of the pressurized lead screw.

Preferably, the monitoring unit may be further equipped with a communication module, thereby being capable of transmitting and receiving data to and from external devices through the communication module.

### Advantageous Effects of the Invention

As described above, according to the present invention, a homogeneous injection device that can consistently maintain the pressure being applied to the plunger of a syringe for homogeneous injection of medicine, may be meticulously and precisely monitored.

Additionally, in case a standard for the injection dosage is determined, the effect of the present invention of regulating the moving distance and pressure of the plunger, when needed, in order to strictly maintain the homogeneousness (or homogeneity or consistency) in the injection dosage, is acknowledged. That is, monitoring may be performed in order to precisely regulate the moving rate of the plunger based on the injection dosage, based on the assumption that a homogeneous quantization of the injection dosage (or flow rate) is not mandatorily determined even if a constant pressure is applied to the plunger.

### DESCRIPTION OF THE DRAWINGS

FIG. 1 is a diagram showing a system for homogeneous injection of medicine according to an exemplary embodiment of the present invention.

FIG. 2 is a diagram showing a device for homogeneous injection of medicine according to an exemplary embodiment of the present invention.

FIG. 3 is a diagram showing a monitoring unit configuring a system for homogeneous injection of medicine according to an exemplary embodiment of the present invention.

FIG. 4 is a diagram showing a gear set and a connection structure of each gear according to an exemplary embodiment of the present invention.

### BEST MODE

Hereinafter, preferred embodiments of the present invention will be described in detail with reference to the appended drawings.

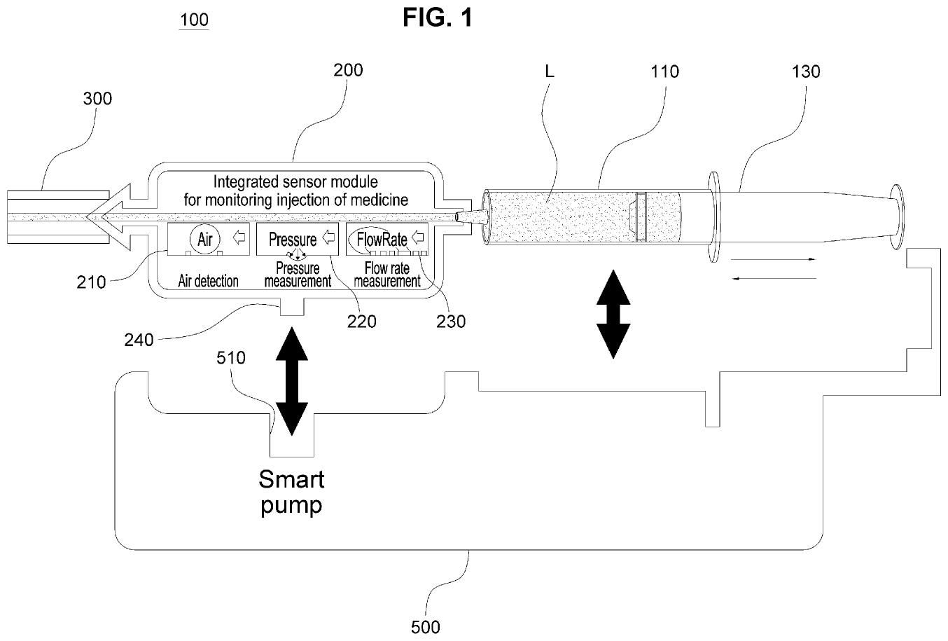

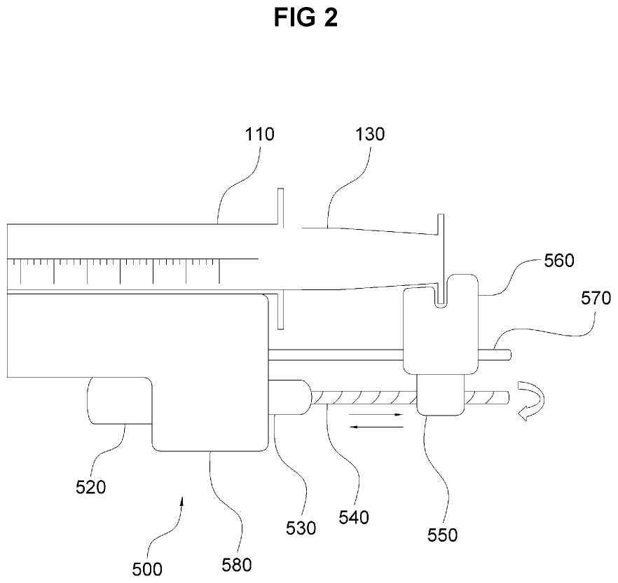

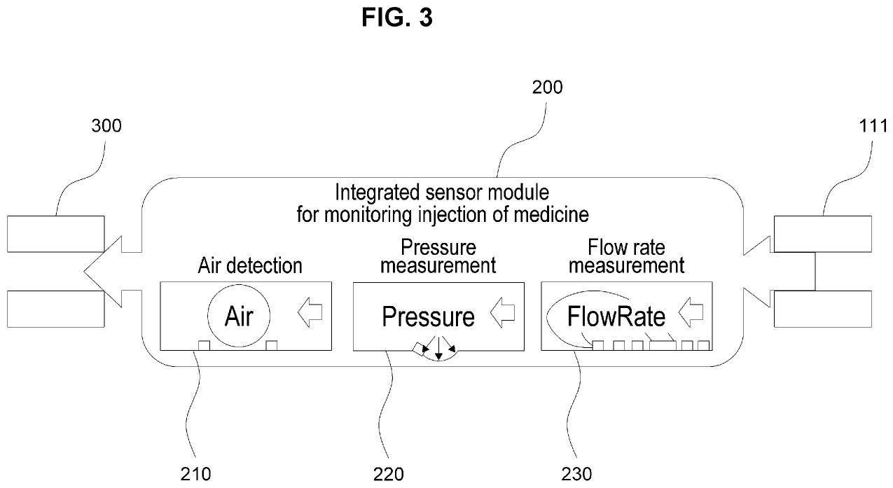

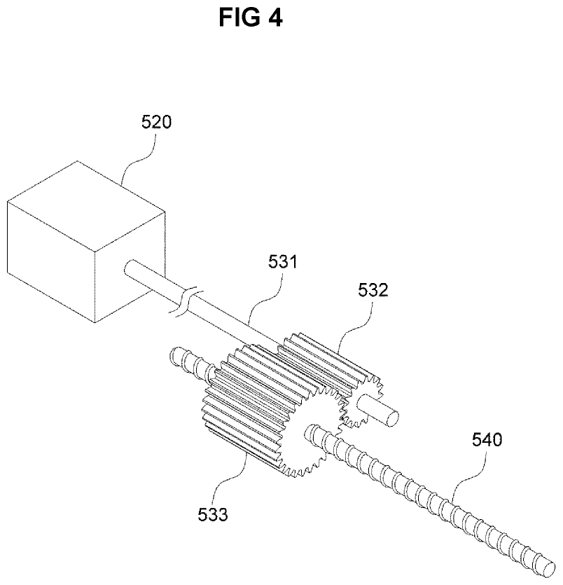

FIG. 1 is a diagram showing a system for homogeneous injection of medicine according to an exemplary embodiment of the present invention. FIG. 2 is a diagram showing a device for homogeneous injection of medicine according to an exemplary embodiment of the present invention. FIG. 3 is a diagram showing a monitoring unit configuring a system for homogeneous injection of medicine according to an exemplary embodiment of the present invention. FIG. 4 is a diagram showing a gear set and a connection structure of each gear according to an exemplary embodiment of the present invention.

A system for homogeneous injection of medicine according to an exemplary embodiment of the present invention includes a monitoring unit 200 monitoring injection dosage of a device for homogeneous injection of medicine 500 and having one side connected to a needle of the syringe and having its opposite side connected to a tube 300, which provides medicine to a patient, so as to monitor the injection dosage of medicine, wherein the device for homogeneous injection of medicine 500 includes a cylinder holding part 580 having a cylinder 110 of a syringe held therein, a plunger resting part 560 being configured to be independent from the cylinder holding part 580 and having an end of the plunger 130 of the syringe rest therein, first and second pressurized lead screw supporting parts 530 and 550 each being provided on one side of the cylinder holding part 580 and one side of the plunger resting part 560, respectively, and being connected to a pressurized lead screw 540 being provided with a screw thread, and a motor 520 being connected to one side of the first pressurized lead screw supporting part 530 so as to rotate the pressurized lead screw 540.

That is, the present invention has organically managed multiple means in order to accurately match the injection dosage of medicine with a configured value. One part is the device for homogeneous injection of medicine 500, and another part is the monitoring unit 200. Up until now, the device for homogeneous injection of medicine has been variously devised, and medicine has been homogeneously injected mostly by relying only on such device for homogeneous injection of medicine.

However, variables are bound to exist during the process of injecting medicine, and an example of such variables may be a device error (malfunction or failure), and another example may be a change in an environment for injecting medicine. A change in the environment for injecting medicine may, for example, refer to in-door ambient temperature, which may vary due to seasonal factors or due to differences in the temperature of a room (or area) where a patient is currently located. Minute differences may occur in the injection dosage (or flow rate) or injection speed of the medicine according to such in-door ambient temperature. However, limitations inevitably exist in the existing injection devices in relation to cancelling such differences and maintaining homogeneous injection. This is because the existing injection devices are managed mostly by consistently maintaining the amount of pressure being applied to plunger 130.

The system for homogeneous injection of medicine 100 according to the present invention is a system for homogeneous injection using a syringe or an intravenous (IV) bag. Herein, a tube 300 being connected to the syringe or IV bag is fixed to the device for homogeneous injection of medicine 500, and the device homogeneously injects the medicine by moving the plunger 130 of the syringe or by squeezing the tube 300 that is connected to the IV bag based on a predetermined injection dosage (or flow rate). Each of the cylinder 110 and the plunger 130, which are parts of the syringe, is supported by two configuration elements each being separated from one another, i.e., the cylinder holding part 580 and the plunger resting part 560, respectively. And, the cylinder holding part 580 and the plunger resting part 560 are connected to one another by a guide 570, thereby allowing the plunger resting part 560 to perform smooth movements along the guide 570 by a predetermined pressure. Thus, pressure is applied to the plunger 130. At this point, the cylinder holding part 580 is in a fixed state and does not move.

In the present invention, the pressurized lead screw 540 that converts rotating movements to linear movements has been applied as a means allowing the movement of the cylinder holding part 580 to be carried out. The pressurized lead screw 540 refers to a screw that operates in order to apply pressure to the plunger 130.

The pressurized lead screw 540 is supported and fixed between the first and second pressurized lead screw supporting parts 530 and 550, each being provided on one side of the cylinder holding part 580 and one side of the plunger resting part 560, respectively, and is connected to the motor 520, which is provided on one side of the first pressurized lead screw supporting part 530, so as to rotate the pressurized lead screw 540 and move the plunger resting part 560. The plunger resting part 560 may apply pressure to the plunger 130 due to the rotation of the pressurized lead screw 540, so that the plunger 130 can be moved inside the cylinder 110 towards a fluid outlet (outlet hole, needle, and so on). And, thus, a homogenous amount of medicine may be discharged (or ejected) from the fluid outlet (outlet hole, needle, and so on) of the syringe.

In the present invention, in order to more homogeneously inject medicine into a patient's body by such homogeneous discharge of medicine, a gear set, which is connected to one side of the motor 520 or the first pressurized lead screw supporting part 530, or which is provided inside the first pressurized lead screw supporting part 530, so as to control the rotation of the pressurized lead screw 540 may be further included.

As shown in FIG. 4, gears of the gear set may be configured of a primary gear (or a main gear) 532 and a secondary gear (or a driven gear) 533. For example, a size of the primary gear 532 and a size of the secondary gear 533 may be configured at a ratio of 1:50 or 1:100, based on the gear size. It will be apparent that the size ratio is merely exemplary, and, therefore, the present invention will not be limited only to the size ratio(s) presented above. At this point, when the primary gear 532 makes a full rotation, the secondary gear 533 makes a 1/50 or 1/100 rotation. Herein, the motor 520 is connected to the primary gear 532, and the pressurized lead screw 540 is connected to the secondary gear 533. The pressurized lead screw 540 rotates according to the rotation of the primary gear 532 and the rotation of the secondary gear 533, wherein the number of rotations may be more accurately controlled than the primary gear 532. Thus, the movement of the plunger resting part 560 according to the rotation of the pressurized lead screw 540 may be more accurately controlled. Therefore, a device for homogeneous injection of medicine 500 having a gear set attached thereto may more homogeneously quantize the injection dosage of medicine than a device for homogeneous injection of medicine 500 that does not have a gear set attached thereto.

In the present invention, by further including a monitoring unit 200, which may monitor injection dosage of medicine, injection speed, pressure size, content of air, and so on, caused by the syringe, the injection dosage (or discharge amount) of medicine may be more homogeneously quantized. Herein, one side of the monitoring unit 200 may be connected to a needle of the syringe or a medicine outlet of the cylinder (110), and its opposite side may be connected to a tube 300, which allows the medicine to be injected into the patient's body through the tube 300. That is, the monitoring unit 200 may monitor various physical quantities of the medicine being discharged from the syringe. And, ultimately, the monitoring unit 200 may control the device for homogeneous injection of medicine 500 by using the monitored results. For example, in the device for homogeneous injection of medicine 500, by controlling the motor 200, so as to limit its number of rotations (or turns), the rotation of the pressurized lead screw 540 and the pressure being applied to the plunger resting part 560 by the pressurized lead screw 540 may be regulated. In order to perform such control of the device for homogeneous injection of medicine 500, the monitoring unit 200 may communication with a control unit, which is equipped to the device for homogeneous injection of medicine 500, so as to allow the control unit to control the operations of the device. That is, the monitoring unit 200 is equipped with a communication module that is capable of transmitting and receiving signal data to and from various external devices.

The monitoring unit 200 may be detachably connected to the device for homogeneous injection of medicine 500 or may be connected to the device for homogeneous injection of medicine 500 in a fixed state.

The monitoring unit 200 may include a pressure measuring unit 220 measuring pressure on the plunger 130 caused by the device for homogeneous injection of medicine 500 or outlet pressure of the medicine, a flow rate measuring unit 230 measuring a flow rate (or injection dosage) of the medicine that is injected by the device for homogeneous injection of medicine 500, and an air measuring unit 210 specifying whether or not air is included in the medicine.

The flow rate (or injection dosage) may also be related to a measurement of the injection speed. As shown in the drawing, the flow rate (or injection dosage) electrodes are formed at both ends of a channel path (connected from an inlet to an outlet) allowing medicine to pass through, and the flow rate (or injection dosage) may be measured by a module that is configured to provide a heater, which connects both electrodes, within a section of the channel. When thermal change is applied to a fluid at each point, and when the temperature is measured accordingly, a thermal transition speed (or rate) changes according to the mass of the fluid. Therefore, the corresponding structure performs a method of calculating the injection dosage (or flow rate) of the medicine, by measuring the temperature of the medicine (or fluid) at each point, converting the change value of the temperature to mass, and then converting the change in mass to speed. Thus, even if the medicine is sensitive to any change in temperature, a change in the injection dosage is monitored, which is then used to control the device for homogeneous injection of medicine 500 by the control unit. Thus, a more precise homogeneous injection of medicine may be carried out. Herein, the flow rate (or injection dosage) may also be related to a movement speed (rate) of the pressurized lead screw 540. That is, the flow rate (or injection dosage) or injection speed of the medicine increases or decreases in proportion to the movement speed (rate) of the pressurized lead screw 540.

The pressure measuring unit 220 may perform measurement from the injection speed (or rate). That is, when it is assumed that a fluid (or liquid) having a predetermined injection speed collides with a virtual surface, the pressure applied to the virtual surface may be converted. Thus, the pressure being applied by the medicine may be measured.

It will be apparent to those skilled in the art that various modifications and variations can be made in this specification without departing from the spirit or scope of this specification. It is also apparent that such variations of this specification are not to be understood individually or separately from the technical scope or spirit of this specification.

What is claimed is:

1. A system for homogeneous injection of medicine, comprising:
a device for homogeneous injection of medicine, wherein the device comprises:
a cylinder holding part having a cylinder of a syringe held therein,
a plunger resting part being configured to be independent from the cylinder holding part and having an end of the plunger of the syringe rest therein,
first and second pressurized lead screw supporting parts each being provided on one side of the cylinder holding part and one side of the plunger resting part, respectively, and being connected to a pressurized lead screw being provided with a screw thread, and
a motor being connected to one side of the first pressurized lead screw supporting part so as to rotate the pressurized lead screw; and
a monitoring unit monitoring injection dosage and having one side connected to a needle of the syringe or a medicine outlet of the syringe cylinder and having its opposite side connected to a tube providing medicine to a patient, thereby monitoring physical quantities related to the injection of the medicine.

2. The system for homogeneous injection of medicine of claim 1, further comprising:
a gear set being connected to one side of the motor or first pressurized lead screw supporting part, or being provided inside the first pressurized lead screw supporting part, so as to control the rotation of the pressurized lead screw.

3. The system for homogeneous injection of medicine of claim 2, wherein rotation of the pressurized lead screw is accurately controlled by configuring the gear set of a primary gear and a secondary gear, wherein a diameter of the secondary gear is greater than a diameter of the primary gear, and by configuring the pressurized lead screw to be connected to the secondary gear, so as to be capable of performing vertical or horizontal movements according to a number of rotations of the secondary gear.

4. The system for homogeneous injection of medicine of claim 1, wherein the monitoring unit is equipped to the device for homogeneous injection of medicine.

5. The system for homogeneous injection of medicine of claim 4, wherein the monitoring unit is connected to a control unit controlling rotation of the motor provided in the device for homogeneous injection of medicine according to a medication injection situation of the device.

6. The system for homogeneous injection of medicine of claim 4, wherein the monitoring unit comprises:
a pressure measuring unit measuring pressure on the plunger caused by the device for homogeneous injection of medicine or outlet pressure of the medicine,
a flow rate measuring unit measuring a flow rate (or injection dosage) of the medicine injected by the device for homogeneous injection of medicine, and
an air measuring unit measuring whether or not air is included in the medicine.

7. The system for homogeneous injection of medicine of claim 4, wherein the monitoring unit is detachably connected to the device for homogeneous injection of medicine.

8. The system for homogeneous injection of medicine of claim 1, wherein the cylinder holding part and the plunger resting part are connected by a guide, and
wherein the plunger resting part moves along a horizontal direction to a vertical shaft of the guide, the guide being used as a medium, according to the rotation of the pressurized lead screw.

9. The system for homogeneous injection of medicine of claim 1, wherein the monitoring unit is further equipped with a communication module, thereby being capable of transmitting and receiving data to and from external devices through the communication module.
